(19) United States
(12) Patent Application Publication
(10) Pub. No.: US 2008/0039471 A1
(43) Pub. Date: Feb. 14, 2008

(54) Composition and method to inhibit tissue plasminogen activator (tPA) - potentiated neurotoxicity

(76) Inventors: Hossein A. Ghanbari, Potomac, MD (US); Zhi-Gang Jiang, Gaithersburg, MD (US)

Correspondence Address: PANACEA PHARMACEUTICALS, INC., 207 PERRY PARKWAY, SUITE 2, GAITHERSBURG, MD, 20877, US

(21) Appl. No.: 11/504,391

(22) Filed: Aug. 14, 2006

(51) Int. Cl. A61K 31/4965; A61K 31/426; A61K 31/4172; A61K 31/44

(52) U.S. Cl. 514/252.1; 514/357; 514/365; 514/400; 514/352

(57) ABSTRACT

The present invention relates to methods of treating, preventing or ameliorating ischemia-related, neural cell degeneration in a subject being treated with a thrombolytic agent, by administering to the subject one or more neuroprotective thiosemicarbazone compounds. More particularly, the present invention relates to methods of preventing, treating or ameliorating the adverse neurological side effects of tissue plasminogen activator, which is used in the treatment of ischemic stroke, by co-administering one or more compounds of the present invention. One such neuroprotective compound is PAN-811. The invention also relates to compositions comprising PAN-811 or analogs thereof in admixture with a thrombolytic agent, such as tPA.

### FIELD OF THE INVENTION

The present invention relates to the discovery that a class of therapeutic thiosemicarbazone compounds, including PAN-811, provides protection of nerve cells after the administration of tissue plasminogen activator (tPA) in the treatment of stroke.

### BACKGROUND OF THE INVENTION

A stroke occurs when the blood supply to part of the brain is suddenly interrupted or when a blood vessel in the brain bursts, spilling blood into the spaces surrounding brain cells. Brain cells die when they no longer receive oxygen and nutrients from the blood or there is sudden bleeding into or around the brain. The symptoms of a stroke include sudden numbness or weakness, especially on one side of the body; sudden confusion or trouble speaking or understanding speech; sudden trouble seeing in one or both eyes; sudden trouble with walking, dizziness, or loss of balance or coordination; or sudden severe headache with no known cause. There are two forms of stroke: ischemic—blockage of a blood vessel supplying the brain, and hemorrhagic—bleeding into or around the brain. The incidence of stroke has increased to 800,000 cases per year in the US in recent years. Stroke has become the third leading cause of death in the US, is a major cause of long-term disability, stroke is a significant burden on public health worldwide.

Generally there are three treatment stages for stroke: prevention, therapy immediately after the stroke, and post-stroke rehabilitation. Acute stroke therapies try to stop a stroke while it is happening by quickly dissolving the blood clot causing an ischemic stroke or by stopping the bleeding of a hemorrhagic stroke. Recombinant tissue-type plasminogen activator (tPA), a thrombolytic-acting drug, is approved as a treatment for ischemic stroke; however, no drug that provides direct neuroprotection is approved for stroke therapy.

Research shows that tPA is physiologically expressed in the adult mouse brain in regions involved in learning and memory (hippocampus), fear and anxiety (amygdala), motor learning (cerebellum), and autonomic and endocrine functions (hypothalamus) (Pawlak et al, “Tissue plasminogen activator in the amygdala is critical for stress-induced anxiety-like behavior.” Nat Neurosci. 2003;6:168-74; Qian et al, “Tissue-plasminogen activator is induced as an immediate-early gene during seizure, kindling and long-term potentiation.” Nature. 1993;361:453-71993; Rodrigues et al., “Molecular mechanisms underlying emotional learning and memory in the lateral amygdala.” Neuron. 2004;44:75-91; Salles et al., “Localization and regulation of the tissue plasminogen activator-plasmin system in the hippocampus.” J Neurosci. 2002;22:2125-342002; Seeds et al., “Tissue plasminogen activator induction in Purkinje neurons after cerebellar motor learning.” Science. 1995;270:1992-4; Seeds et al., Neuronal migration is retarded in mice lacking the tissue plasminogen activator gene. Proc Natl Acad Sci USA. 1999;96:14118-23; Seeds et al., “Absence of tissue plasminogen activator gene or activity impairs mouse cerebellar motor learning.” J Neurosci. 2003;23:7368-75; Teesalu et al, “Tissue plasminogen activator and neuroserpin are widely expressed in the human central nervous system.” Thromb Haemost. 2004;92:358-68). When tPA is expressed in neurons it has a beneficial role in facilitating neurite outgrowth and pathfinding through the processing of extracellular matrix proteoglycans (for review, see Tsirka, “Tissue plasminogen activator as a modulator of neuronal survival and function.” Biochem Soc Trans. 2001).

Intravenous infusion of tPA, such as recombinant tPA and its derivatives, is used for lysis of fibrin clots, which helps restore blood flow following myocardial infarction or ischemic stroke (Collen, Ham-Wasserman lecture: Role of the plasminogen system in fibrin-homeostasis and tissue remodeling. Hematology (Am Soc Hematol Educ Program). 2001;:1-9). tPA is a serine protease that cleaves a specific peptide bond within the zymogen, plasminogen, to generate the active protease, plasmin, which is capable of degrading numerous substrates. In the vasculature, plasmin efficiently breaks down fibrin clots.

In the treatment of ischemic stroke, tPA and other thrombolytic agents have been shown to have neurotoxic side effects. For instance, plasminogen activators have been shown to be N-methyl-D-aspartic acid (NMDA) receptor agonists. Stimulation of the NMDA receptor can impair brain function. Moreover, tPA can produce bleeding in the brain, which itself can produce further neurological damage.

Although systemic administration of tPA to treat ischemic stroke plays an important function in acute therapy by dissolving the fibrin clot, it will also infiltrate into extracellular spaces through an interrupted and compromised blood-brain barrier (BBB), and act to robustly potentiate ischemic neurodegeneration. In this way, tPA functions as an agonist to stimulate a cell surface receptor on microglia (the macrophage-like immunocompetent cells of the central nervous system) and results in their activation. Once activated after neuronal injury, microglia contribute to the ensuing neurodegeneration (Tsirka, supra). This pathological effect of tPA mainly involves mediating a critical step in the progression of excitotoxin-induced neurodegeneration and causes extra calcium influx into neurons. Because of these adverse effects, tPA infusion must be administered within 3 hours following the onset of ischemic stroke in order to minimize severe adverse side effects.

Various strategies have been recently developed to ensure therapeutic activity and avoid this adverse effect of tPA for ischemic stroke therapy. For example, a synthetic tPA-inhibitor, 2,7-bis-(4-amidino-benzylidene)-cycloheptan-1-one dihydrochloride (tPA-Stop™), has been reported as effective in reducing tPA-potentiated, NMDA-mediated, excitotoxic neuronal death. However, this compound fails to modulate alpha-amino-2,3-dihydro-5-methyl-3-oxo-4-isoxazole propanoic acid or kainate-mediated necrosis. This indicates that tPA-Stop™ acts at the receptor level (Liot et al., 2,7-Bis-(4-amidinobenzylidene)-cycloheptan-1-one dihydrochloride, tPA stop, prevents tPA-enhanced excitotoxicity both in vitro and in vivo. J Cereb Blood Flow Metab. October 2004;24(10):1153-9).

tPA's neurovascular toxicity to ischemic human brain endothelium and mouse cortical neurons is blocked by activated protein C (APC) by inhibiting tPA-induced caspase-8 activation of caspase-3 in endothelium, and caspase-3-dependent nuclear translocation of apoptosis-inducing factor in NMDA-treated neurons and reduced tPA-mediated cerebral ischemic injury in mice (See Liu et al., Tissue plasminogen activator neurovascular toxicity is controlled by activated protein C. Nat Med. 2004;10(12):1295-6). However, given the high molecular weight of APC (56000 Daltons), passage through the BBB is hampered, a necessity for protection from tPA-potentiated neurotoxicity in the central nervous system (CNS).

Since tPA-potentiated neurotoxicity is excitatory, via increasing calcium influx, a small molecule that could easily infiltrate the brain and that has the ability to eliminate (or suppress) intracellular accumulation of free calcium, for instance, would be highly advantageous in protecting neurons and preventing tPA's adverse side effects on the CNS.

Moreover, it is becoming apparent that the therapeutic administration of tPA for other ailments, such as pulmonary embolism, central venous catheter occlusions, and myocardial infarction, may also cause excitatory neurotoxicity in the CNS, because tPA causes permeability of the of the BBB, allowing it to pass into the brain and exert its deleterious effects. Thus, neuroprotection would be beneficial under any circumstances tPA is being administered to a patient.

Therapy with such a neuroprotective agent initiated before or concurrently or after with tPA could serve to extend the therapeutic window of tPA by blocking it's adverse side effects on salvageable nerve tissue. Moreover, administration of the neuroprotective agent after initiation of thrombolytic treatment may enhance the delivery of the neuroprotective agent to the brain, given that tPA has been shown to increase vascular permeability, which is thought to likely initiate opening of the BBB. (Yepes, M. et al., “Tissue-type plasminogen activator induces opening of the blood-brain barrier via the LDL receptor-related protein”, J. Clin. Invest., 112:10, pages 1533-1540 (2003)).

Studies by the present inventors have demonstrated that a class of thiosemicarbazone compounds, such as PAN-811 (also known as 3-AP, or 3-aminopyridine-2-carboxaldehyde thiosemicarbazone), effectively inhibits ischemic neurodegeneration, hypothetically via chelating intracellular free calcium. The present inventors have now discovered that PAN-811 and analogues thereof can also act to suppress the tPA-potentiated excitatory neurotoxicity that occurs when administered during ischemic stroke or glutamate insult. While not being bound to a particular theory of action, it is believed this class of thiosemicarbazones is effective in this manner because it may interfere with the influxed intracellular calcium (the excitatory neurotoxicity) caused by tPA.

### SUMMARY OF THE INVENTION

The present invention is directed to a new use of certain N-heterocyclic carboxaldehyde thiosemicarbazones (HCTs), which have been known as useful as antineoplastic agents, acting as potent inhibitors of ribonucleotide reductase, as well as neuroprotectants. Methods of treatment of tumors using such compounds are disclosed inter alia in U.S. Pat. Nos. 5,721,259 and 5,281,715 of Sartorelli et al. Methods of using such compounds for neuroprotection are disclosed in US Patent Publication No. 20060160826, which is hereby incorporated by reference in its entirety.

The present invention is directed to methods of using such compounds in conjunction with the tPA infusion therapy that is used in the treatment of ischemic stroke as well as other related ischemic conditions.

### BRIEF DESCRIPTION OF THE DRAWINGS

FIG. 1 shows the results of LDH (FIG. 1A) and MTS (FIG. 1B) assays in Example 1, and represents a potentiated effect of tPA on glutamate-induced neuronal cell death.

FIG. 2 shows the results of an LDH assay in Example 1, and represents the protective effects of PAN-811 on tPA/glutamate-induced neuronal cell death.

FIG. 3 shows the results of LDH (FIG. 3A) and MTS (FIG. 3B) assays in Example 2, and represents a potentiated effect of tPA on hypoxia/hypoglycemia (H/H)-induced neuronal cell death.

FIG. 4 shows the results of an LDH assay in Example 2, and represents the protective effects of PAN-811 on tPA/H/H-induced neuronal cell death.

### DETAILED DESCRIPTION OF THE INVENTION

The present invention relates to methods of preventing, ameliorating and/or treating the neurodegeneration that ensues as a result of therapeutically administered tPA, which comprises the co-administration to the patient being treated one or more N-heterocyclic 2-carboxaldehyde thiosemicarbazones (HCTs), or pharmaceutically acceptable salts or prodrugs thereof: Such useful compounds are encompassed by Formula I:

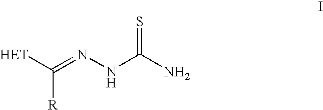

### In a preferred embodiment, the compound is of Formula II:

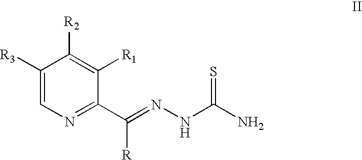

where R is H or C₁-C₄-alkyl; and R₁, R₂ and R₃ are independently selected from H and amino.

In another preferred embodiment, the compound is of Formula III:

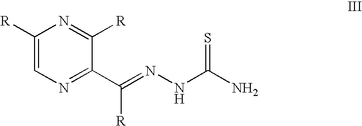

where R is H or C₁-C₄-alkyl; and R₁ and R₂ are independently selected from H and amino.

In another preferred embodiment, the compound is of Formula IV:

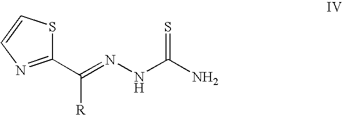

where R is H or C₁-C₄-alkyl.

Yet another preferred embodiment is a compound of formula V:

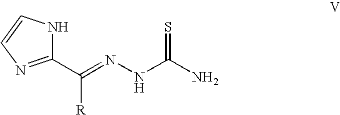

where R is R is H or C₁-C₄-alkyl.

Finally, another preferred embodiment is a compound of Formula VI:

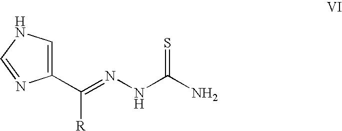

where R is H or C₁-C₄-alkyl.

As more preferred embodiments, the compounds of the present invention are selected from:

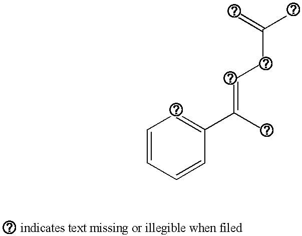

(of Formula II, where R is methyl, and R₁, R₂ and R₃ are H.)

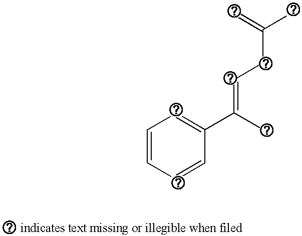

(of Formula III, where R is methyl and R₁ and R₂ are H.)

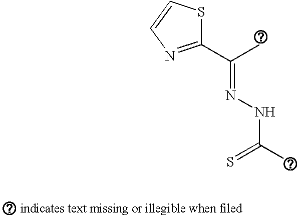

(of Formula IV, where R is methyl)

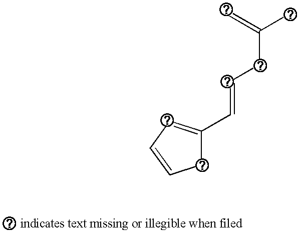

(of Formula IV, where R is H)

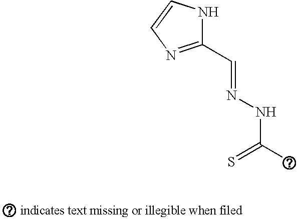

(of Formula V, where R is H) and

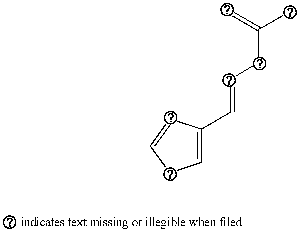

(of Formula VI, where R is H).

A most preferred embodiment of the present invention relates to methods of preventing or treating the undesirable neurotoxic side effect of tPA by co-administering to the stroke patient PAN 811 (3-aminopyridine-2-carboxaldehyde thiosemicarbazone) having the formula:

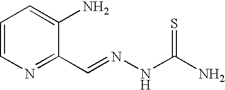

Certain compounds encompassed by the present invention may exist as E, Z-stereoisomers about the C═N double bond and the invention includes the mixture of isomers as well as the individual isomers that may be separated according to methods that are well known to those of ordinary skill in the art. Certain compounds of the present invention may exist as optical isomers and the invention includes both the racemic mixtures of such optical isomers as well as the individual entantiomers that may be separated according to methods that are well known to those of ordinary skill in the art.

The means for synthesis of compounds useful in the methods of the invention are well known in the art. Such synthetic schemes are described in U.S. Pat. Nos. 5,281,715; 5,767,134; 4,447,427; 5,869,676; and 5,721,259, all of which are incorporated herein by reference in their entireties.

Examples of pharmaceutically acceptable salts are inorganic and organic acid addition salts such as hydrochloride, hydrobromide, phosphate, sulphate, citrate, lactate, tartrate, maleate, fumarate, acetic acid, dichloroacetic acid and oxalate.

Examples of prodrugs include, for example, esters of the compounds with R₁—R₃ as hydroxyalkyl, and these may be prepared in accordance with known techniques.

PAN-811 is a hydrophobic small molecule (MW 195 Daltons) with a calcium chelating function, which will pass the BBB, demonstrated by systematic administration (Jiang et al., A multifunctional cytoprotective agent that reduces neurodegeneration after ischemia. Proc Natl Acad Sci USA. 2006; 103(5):1581-6.). It is surprising and unexpected that the inventors discovered that the compound, 3-aminopyridine-2-carboxaldehyde thiosemicarbazone (PAN-811), and analogs thereof, is effective in combating the neurotoxic side effect of tPA.

By the terms “co-administration” or “co-administering” in this context is meant that the neuroprotective drugs of the present invention are given substantially contemporaneously, either simultaneously or sequentially, with thrombolytic therapy (e.g., by tPA). By “sequentially” is meant that either the neuroprotective agent is given prior to, or after, administration of the thrombolytic agent; however, such a mode of administration includes the possibility of both therapeutic agents being present in the body at the same time. The present invention includes a method of administering the neuroprotective drug just prior to initiation of, or directly following, thrombolytic treatment, and can be in a single dose, multiple doses, or continuous infusion. All of these embodiments are considered encompassed by the co-administration terminology.

Determination of a therapeutically or prophylactically neuroprotective amount of the compounds of the present invention can be readily made by the clinician by the use of known techniques and by observing results obtained under analogous circumstances. The dosages may be varied depending upon the requirements of the patient in the judgment of the clinician and the severity of the condition being treated. In determining the effective neuroprotective amount or dose, a number of factors are considered by the clinician, such as: the specific cause of the ischemic or hypoxic state and its likelihood of recurring or worsening; the mode and route of administration; the desired time course of treatment; the species of mammal; its size, age, and general health; the response of the individual patient; and other relevant circumstances.

The neuroprotective agent is present in a pharmaceutical composition of the present invention (i.e., one or more of the thiosemicarbazones), or in a course of emergency ischemic treatment, in a therapeutically or prophylactically effective amount. By a “therapeutically effective amount” is meant an amount effective, at dosages and for periods of time necessary, to achieve the desired therapeutic result of positively influencing the course of a particular disease state. Of course, therapeutically effective amounts of the active agent(s) may vary according to factors such as the disease state, age, sex, and weight of the individual, and the ability of the agent to elicit a desired response in the individual. Dosage regimens may be adjusted to provide the optimum therapeutic response. A therapeutically effective amount is also one in which any toxic or detrimental effects of the agent are outweighed by the therapeutically beneficial effects. By a “prophylactically effective amount” is meant an amount effective, at dosages and for periods of time necessary, to achieve the desired prophylactic result. Typically, since a prophylactic dose is used in subjects prior to or at an earlier clinical stage, the prophylactically effective amount may be less than the therapeutically effective amount.

It is contemplated that the pharmaceutical compositions of the present invention will contain (in addition to a thrombolytic agent) PAN-811, or an analog or combination thereof, in amounts suitable for a dosage regimen of about the same as or, more preferably less than, those presently employed in antineoplastic treatment with 3-aminopyridine-2-carboxaldehyde thiosemicarbazone (aka, Triapine®, Vion Pharmaceuticals, Inc.). For instance, doses less than 60 mg/m² and higher doses of 60 and 80 mg/m² of 3-aminopyridine-2-carboxaldehyde thiosemicarbazone (‘PAN-811’) using a single intravenous dosing schedule (infusion for 8 h) have been used in clinical trials for cancer therapy. 3-aminopyridine-2-carboxaldehyde thiosemicarbazone has also been administered by single 2-h infusion every 4 weeks in 46 courses of therapy. It is well tolerated at doses up to 105 mg/m² and a dose limiting toxicity was not identified. Based on the studies in the Examples as well as previous work by the present inventors, the effective dose of PAN-811 for neuroprotection appears to be far below its maximal tolerated dose, as well as being well below the dosages typically used for cancer treatment. It is contemplated that the disclosed analogs have similar pharmacodynamic profiles as that of PAN-811, and thus the dosage regimens will be the same or similar to PAN-811 for neuroprotection. The amount of thrombolytic agent in the compositions is preferably that which is currently used in treatment of ischemic stroke or cardiac ischemia, and may depend on which of these conditions is being treated. Moreover, since administration of tPA is of limited duration, the co-administration of the neuroprotective agent may allow a somewhat longer treatment phase with tPA. Treatment may be initiated with smaller dosages, which are less than the optimum dose of the agent. Thereafter, the dosage may be increased by small increments until the optimum effect under the circumstances is reached. For convenience, the total dosage may be divided and administered in portions during a course of time if desired.

One aspect of the present invention is directed to pharmaceutical compositions of the 2-carboxyaldehyde thiosemicarbazones useful in the methods of the invention together with a thrombolytic agent, in a pharmaceutically acceptable carrier or diluent. The pharmaceutical compositions of the invention may comprise one or more of the thiosemicarbazones, and may also comprise other therapeutic agents.

Representative thrombolytic agents include, for example, alteplase (tissue plasminogen activator (tPA)), anistreplase, reteplase, urokinase, and streptokinase. Since tPA is the only approved thrombolytic agent for stroke, this is the preferred embodiment.

As used herein “pharmaceutically acceptable carrier or diluent” includes any and all solvents, dispersion media, coatings, antibacterial and antifungal agents, isotonic and absorption delaying agents, and the like that are physiologically compatible. The type of carrier can be selected based upon the intended route of administration. In various embodiments, the carrier is suitable for intravenous, intraperitoneal, subcutaneous, intramuscular, topical, transdermal or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated.

As a preferred embodiment, the carrier is one suitable for intravenous administration. Sterile intravenous solutions can be prepared by incorporating a desired amount of the active compound or compounds in a pharmaceutically acceptable liquid vehicle and filter sterilized. Generally, dispersions are prepared by incorporating the active compound(s) into a sterile vehicle containing a basic dispersion medium. In the case of sterile powders for the preparation of sterile injectable solutions, the preferred methods of preparation are vacuum drying and freeze-drying, which will yield a powder of the active ingredient plus any additional desired ingredient from a previously sterile-filtered solution thereof.

The methods and pharmaceutical compositions of the invention are used for any animal that is being treated with a thrombolytic agent, particularly tPA, and thus in need of the beneficial neuroprotective effects of the compounds disclosed. Preferably the animal is a mammal, and most preferably, human. Preferably the methods and compositions are used to treat a human patient experiencing an ischemic event, such as ischemic stroke or cardiac ischemia or any ischemia related condition in which a thrombolytic agent like tPA is used in its treatment. Preferably, the patient is one in which ischemic stroke symptoms have occurred within about 3 hours of the initiation of treatment.

This invention is further illustrated by the following examples, which are not intended to limit the present invention. The contents of all references, patents, and published patent applications cited throughout this application are specifically and entirely incorporated herein by reference.

### EXAMPLES

### Example 1

### PAN-811 Suppresses tPA-Potentiated Neurotoxicity Resulted from Glutamate Insult

For this example PAN-811 was dissolved in PEG:EtOH (7:3) to a concentration of 25 mM, and further diluted in neurobasal medium to a final concentration of 2 μM. The control vehicle is 1:12,500 PEG:EtOH (7:3 in BSS).

A mixed culture of embryonic (E18) rat cortical and striatal neurons was cultured for 22 days in 50% antioxidative (AO) neurobasal medium (made by replacing 50% of the neurobasal culture medium with a medium comprised of neurobasal plus B27 supplement (minus AO, i.e., without the antioxidants vitamin E, vitamin E acetate, 2.5 mg/L superoxide dismutase, 2.5 mg/L catalase, and 1 mg/L glutathione) (from Invitrogen)), and then divided into test groups in accordance with the following scheme: (1) not insulted (control); (2) insulted with 15 μM glutamate; (3) insulted with 20 μg/ml tPA; or (4) insulted with both 15 μM glutamate and 20 μg/ml tPA, for a period of 24 hours. In a further test category, neurons that were to be insulted for 24 hours with the combination of 15 μM glutamate and 20 μg/ml tPA, were divided into groups in which (1) cells were not pre-treated prior to insult; (2) were pretreated overnight with 1:12,500 vehicle (used for PAN-811), or (3) were pre-treated overnight with PAN-811 (in vehicle).

The experimental results were evaluated by morphological changes, lactate dehydrogenase (LDH) and MTS (mitochondrial function) analyses. Morphologically, neurons in the ‘no insult/no pretreatment’ control group showed intact cell soma with brilliant phase contrast outline, emitting neurites (or processes), which formed a network background. In the tPA treated group, neurons remained healthy. In contrast, glutamate insult resulted in strong neuronal cell death, where most of the neurons were fragmented, although some intact neurons were still observed in the vision field. In the group where neurons were co-treated with glutamate and tPA, the neurons formed small pieces of fragments with none of them surviving.

The lactate dehydrogenase (LDH) assay (Promega) is based on the reduction of NAD by the action of LDH. The resulting reduced NAD (NADH) is utilized in the stoichiometric conversion of a tetrazolium dye. If cell-free aliquots of medium from cultures given different treatments are assayed, then the amount of LDH activity can be used as an indicator of relative cell death as well as a function of membrane integrity. A 50 μl aliquot of culture medium from a well in tested 96-well plate is transferred into a well in unused plate and supplemented with 25 μl of equally-mixed Substrate, Enzyme and Dye Solutions (Sigma). The preparation is incubated at room temperature for 20-30 minutes, and then measured spectrophotometrically at wavelength of 490 nm.

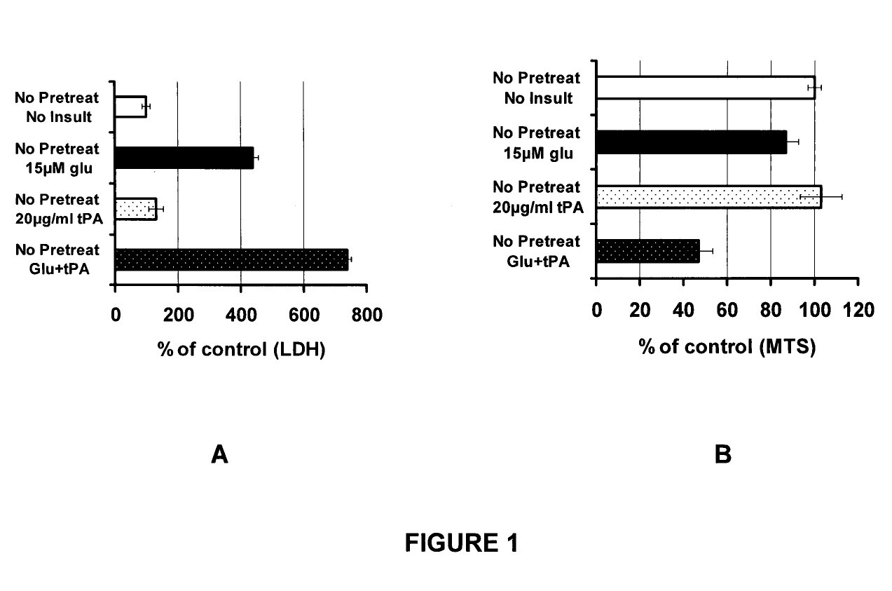

The LDH assays demonstrated that tPA was not toxic to neurons by itself, while glutamate at a concentration of 15 μM resulted in intermediate neuronal cell death, demonstrated by a 4.4-fold increase in LDH over the control. Severe neuronal cell death occurred when treated with a combination of glutamate and tPA, in which the LDH reading leaped to 7.4-fold over the control. See FIG. 1A.

The 3-(4,5-dimethylthiazol-2-yl)-5-(3-carboxymethoxyphenyl)-2-(4-sulfophenyl)-2H-tetrazolium (MTS) assay (Promega) is routinely used to evaluate mitochondrial function, and reduced readings are correlated with neuronal cell death. The MTS tetrazolium compound is reduced in metabolically active mitochondria into a colored formazan product that is soluble in tissue culture medium, and can be detected via its absorbance 490 nm. 10 μl of MTS reagent (Promega) are added to each well of the 96 well assay plates containing the samples in 50 μl of culture medium. The plate is then incubated in a humidified, 5% CO₂ atmosphere at 37° C. for 1-2 hours until the color is fully developed. The absorbance at 490 nm was recorded using a Bio-Rad 96 well plate reader.

tPA alone did not cause any reduction in the MTS reading, indicating little if any influence on mitochondrial function. Glutamate at a concentration of 15 μM resulted in a slight reduction (13%) in the MTS reading, while a combination of glutamate and tPA resulted in a great reduction in mitochondrial function, showing a 53% reduction in the MTS assay reading over the control. See FIG. 1B.

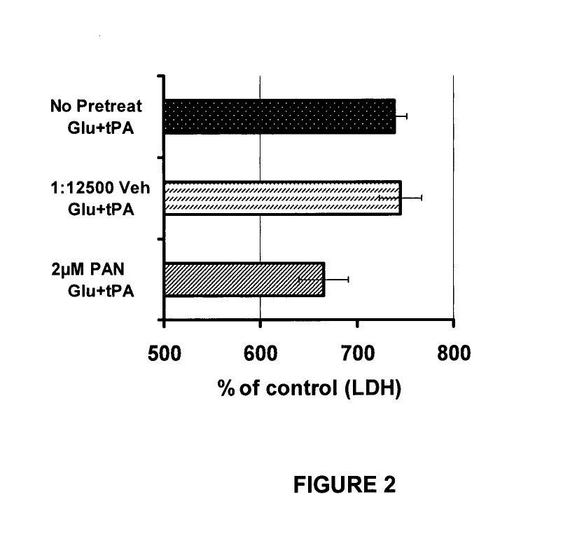

If the test group that was insulted with a combination of tPA and glutamate is pre-treated with 2 μM PAN-811, the LDH reading was reduced by about 11% as compared to no such pre-treatment or pre-treatment with control vehicle alone, indicating a protective effect of PAN-811 on the neurons (FIG. 2).

### Example 2

### PAN-811 Significantly Inhibits tPA-Potentiated Neurotoxicity Resulting from Ischemic Insult

In this example, PAN-811 was dissolved in PEG:EtOH (7:3) to a concentration of 25 mM, and further diluted in neurobasal medium to final concentrations of 1.25, 2.5, 5 and 10 μM. The control vehicle is 1:10,000 PEG:EtOH (7:3 in BSS).

A mixed culture of embryonic (E18) rat cortical and striatal neurons was cultured for 13 days in neurobasal medium (Invitrogen), then insulted by hypoglycemia/hypoxia (H/H) conditions for 6 hours, or a combination of H/H and 20 μg/ml tPA. The H/H conditions were 1.2 mM glucose (normally 25 mM in culture medium) and the absence of oxygen for 6 hours. (Following H/H conditions, the cultures were returned to the typical conditions of 25 mM glucose medium and 95% ambient air plus 5% CO₂.)

In a further experiment, cells that were under H/H conditions and also treated with the tPA were divided into groups, which were either co-treated or post-treated (i.e., during or after the 6 hour insult period) with control vehicle or each of PAN-811 at concentrations of 1.25, 2.5, 5 and 10 μM.

The experimental results were evaluated by morphological changes, and LDH and MTS analyses. Morphologically, by 17 hours post-insult, the combination of H/H and tPA resulted in robust neuronal cell death, while H/H alone only caused an intermediate neurotoxicity.

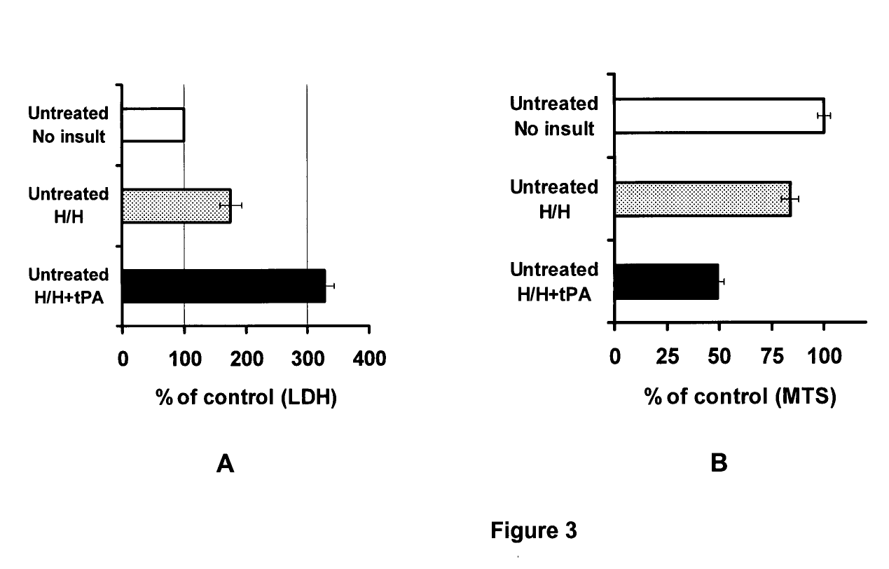

In microscopic detail, neurons did not show obvious morphological changes by the end of the 17-hour H/H insult, whereas neurons treated with a combination of H/H and tPA formed small pieces of fragmentation. Quantitatively, H/H alone resulted in a 1.8-fold increase in LDH level over control. The combination H/H and tPA insult showed a 3.3-fold enhancement in the LDH reading. See FIG. 3A.

On the other hand, H/H alone reduced the MTS reading by 14%, whereas the combination insult decreased MTS reading by 51%. See FIG. 3B.

Both the LDH and the MTS results indicate that tPA greatly potentiates ischemic neurotoxicity.

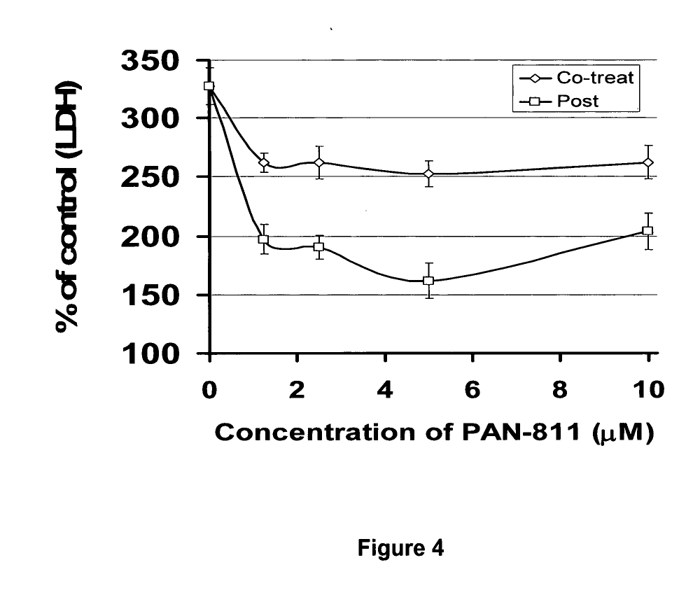

Co- or post-treatment with varying concentrations of PAN-811 preserved neuronal morphology under H/H and tPA conditions. Quantitatively, this protection showed a dose response correlation, whether PAN-811 was administered at same time as the start of insult (co-treatment) or at termination of the insult (post-treatment). For example, the LDH reading decreased by 23% when neurons were co-treated with 5 μM PAN-811. When neurons were treated with 5 μM PAN-811 post-insult, it resulted in 51% protection. See FIG. 4. In fact, a concentration of PAN-811 as low as 1.25 μM shows neuroprotection as this was effective in significantly reducing LDH readings.

Those of skill in the art will readily appreciate that the present invention is well adapted to carry out the objects and obtain the ends and advantages mentioned, as well as those inherent therein, and would know that various modifications and variations can be made in practicing the present invention without departing from the spirit or scope of the invention as claimed in the appended claims. Such modifications and variations are considered by the inventors as encompassed within the invention as a whole and as claimed herein.

What is claimed is:

1. A method for preventing, ameliorating or treating the adverse neurological effects of a therapeutically administered thrombolytic agent in a subject, comprising co-administering to the subject an effective amount of one or more compounds of Formula I, or pharmaceutically acceptable salts or prodrugs thereof: 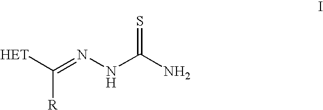
where HET is a 5 or 6 membered heteroaryl residue having 1 or 2 heteroatoms selected from N and S, and optionally substituted with an amino group; and R is H or C₁-C₄-alkyl.

2. The method of claim 1, wherein the compound, or a salt or prodrug thereof, administered to the subject is of Formula II: 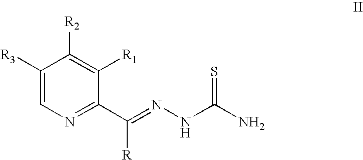
where R is H or C₁-C₄-alkyl; and R₁, R₂ and R₃ are independently selected from H and amino.

3. The method of claim 1, wherein the compound, or a salt or prodrug thereof, administered to the subject is of Formula III: 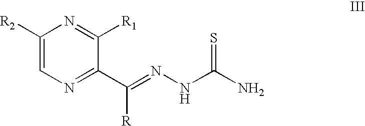
where R is H or C₁-C₄-alkyl; and R₁ and R₂ are independently selected from H and amino.

4. The method of claim 1, wherein the compound, or a salt or prodrug thereof, administered to the subject is of Formula IV: 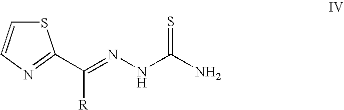
where R is H or C₁-C₄-alkyl.

5. The method of claim 1, wherein the compound, or a salt or prodrug thereof, administered to the subject is of Formula V: 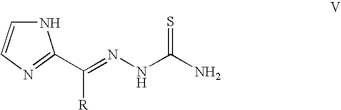
where R is H or C₁-C₄-alkyl.

6. The method of claim 1, wherein the compound, or a salt or prodrug thereof, administered to the subject is of Formula VI: 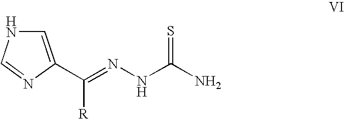
where R is H or C₁-C₄-alkyl.

7. The method of claim 1, wherein the compound, or a salt or prodrug thereof, administered to the subject is

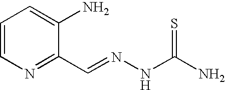

8. The method of claim 2, wherein R is methyl and R₁, R₂ and R₃ are H.

9. The method of claim 3, wherein R is methyl and R₁ and R₂ are H.

10. The method of claim 4, wherein R is methyl.

11. The method of claim 5, wherein R is H.

12. The method of claim 6, wherein R is H.

13. The method of claim 1, wherein the thrombolytic agent is a tissue plasminogen activator.

14. The method of claim 1, wherein the subject is a human.

15. The method of claim 1, wherein the at least one compound is administered prior to initiation of thrombolytic treatment.

16. The method of claim 1, wherein the at least one compound is administered following termination of thrombolytic treatment.

17. The method of claim 15, wherein the at least one compound is administered as a single dose.

18. A pharmaceutical composition comprising one or more of the compounds according to claim 1, together with a thrombolytic agent and a pharmaceutically acceptable carrier.

19. The composition of claim 18, wherein the compound is PAN-811 and the thrombolytic agent is a tissue plasminogen activator.

20. The composition of claim 19, which is in the form of a sterile injectable, intravenous or intra-arterial solution.
